(19) United States
(12) Patent Application Publication
(10) Pub. No.: US 2021/0225489 A1
(43) Pub. Date: Jul. 22, 2021

(54) DETERMINING THE LIKELIHOOD OF PATIENT SELF-EXTUBATION

(71) Applicant: KONINKLIJKE PHILIPS N.V., EINDHOVEN (NL)

(72) Inventors: Cornelis Conradus Adrianus Maria van Zon, Cambridge, MA (US); Francesco Vicario, Cambridge, MA (US); Nikolaos Karamolegkos, Cambridge, MA (US); HAIBO WANG, CAMBRIDGE, MA (US)

(21) Appl. No.: 17/092,388

(22) Filed: Nov. 9, 2020

Related U.S. Application Data
(60) Provisional application No. 62/964,546, filed on Jan. 22, 2020.

(51) Int. Cl. G16H 20/40; G16H 40/63

(52) U.S. Cl. CPC G16H 20/40; G16H 40/63

(57) ABSTRACT

The invention discloses a computer-implemented method for monitoring an intubated patient (104), the method comprising receiving, from one or more sensors (110, 112) associated with the intubated patient, data relating to the intubated patient; determining a likelihood of self-extubation by the intubated patient based on the received data; and responsive to determining that the likelihood of self-extubation is greater than a defined threshold, generating an alert signal.

### CROSS-REFERENCE TO RELATED APPLICATIONS

This patent application claims the priority benefit under 35 U.S.C. § 119(e) of U.S. Provisional Application No. 62/964,546 filed on Jan. 22, 2020, the contents of which are herein incorporated by reference.

### FIELD OF THE INVENTION

The invention relates to determining a likelihood that an intubated patient will self-extubate.

### BACKGROUND OF THE INVENTION

Mechanical ventilation may be used within an intensive care unit when a patient is unable to breath on their own. A widely used mechanical ventilation technique is invasive ventilation, which provides access to the patient's lower airways through tracheostomy or an endotracheal tube. In addition to ventilation management during the course of mechanical ventilation, extubation (i.e. the removal of the tube) is considered a critical component for a successful therapy. If reintubation after extubation is required, patient recovery may be adversely affected. Adverse effects include a patient requiring mechanical ventilation for a longer period of time, an extended length of hospital stay, and/or an increased risk of further medical complications such as nosocomial pneumonia.

Planned extubation refers to weaning a patient from mechanical ventilation and removal of the endotracheal tube as planned by a medical team following well-established procedures. Nevertheless, unplanned extubations, whether accidental due to hospital personnel's inappropriate manipulation of the endotracheal tube or deliberate due to the patient's action, are relatively frequent events in intensive care units.

Prevention of unplanned extubation, including self-extubation by a patient, requires regular surveillance of the patient by a medical team. Continuous bedside presence, or even remote surveillance, of the patient by a member of the medical team is a major challenge for clinical institutions. It is known that systems exist which determine when a patient has removed the tube from their airway (i.e. performed self-extubation), but such systems are capable of alerting medical personnel only after the event of self-extubation. Therefore, it would be desirable to be able to determine a likelihood that an intubated patient will self-extubate before the self-extubation event occurs.

### SUMMARY OF THE INVENTION

Patients who are undergoing assisted ventilation may require an endotracheal tube in order to enable them to breath. There are a number of possible negative consequences associated with a patient who attempts to remove their endotracheal tube, which include an increased length of time required on assisted ventilation, an extended length of hospital stay, and/or an increased risk of further medical complications such as nosocomial pneumonia. Predicting when a patient will remove their endotracheal tube, or self-extubate, is therefore of importance in order to maximize the patient's wellbeing and to minimize the resources and expense associated with the patient's care. Embodiments disclosed herein provide a mechanism which enables such a prediction to be made, so that appropriate action may be taken in the event that a self-extubation event is deemed likely to occur.

According to a first aspect, the present invention provides a computer-implemented method for monitoring an intubated patient, the method comprising receiving, from one or more sensors associated with the intubated patient, data relating to the intubated patient; determining a likelihood of self-extubation by the intubated patient based on the received data; and responsive to determining that the likelihood of self-extubation is greater than a defined threshold, generating an alert signal.

The embodiments of the present disclosure allow a patient to be monitored, and a likelihood of the patient removing their endotracheal tube determined, continuously, with no input necessarily required from a medical professional until an alert has been generated indicating that the likelihood of the patient self-extubating has exceeded a predefined threshold value. Advantageously, the present disclosure allows medical professionals to attend to other duties as opposed to monitoring intubated patients for signs of discomfort, thereby reducing pressure on the medical professionals' time and allowing them to attend to other, potentially critically ill, patients requiring urgent care. In the absence of the present disclosure, if a medical professional is required elsewhere, such as to attend to an emergency, they may not be able to assess whether an intubated patient is likely to self-extubate or not. A further advantage of the present disclosure is that patient discomfort and, therefore, the likelihood of a patient self-extubating, may be assessed quantitatively, as opposed to a medical professional making a subjective assessment, which may improve the reliability of pre-emption of patient self-extubation. Improving patient monitoring and the reliability of pre-emption of patient self-extubation may thereby improve patient outcomes, which may lead to, for example, less time required on assisted ventilation, shorter hospital stays and/or lower hospitalization costs.

In some embodiments, determining the likelihood of self-extubation by the intubated patient may comprise analyzing a facial expression of the intubated patient; and determining, based on said analysis, that a particular facial expression of the intubated patient is indicative of an increased likelihood of self-extubation.

Determining the likelihood of self-extubation by the intubated patient may, in some embodiments, comprise measuring a motion of the intubated patient and/or a motion of a piece of equipment used in the intubation; and responsive to determining that the measured motion exceeds a defined movement threshold, determining that the measured movement is indicative of an increased likelihood of self-extubation.

In some embodiments, measuring a motion of the intubated patient may comprise measuring a motion of one or more of a hand, an arm, a foot, a leg, the head and the torso of the intubated patient.

Determining a likelihood of self-extubation by the intubated patient may, in some embodiments, comprise providing the received data as an input to a trained predictive model, wherein the predictive model is trained to determine the likelihood of self-extubation based on the input data.

In some embodiments, determining a likelihood of self-extubation by the intubated patient may comprise applying a set of rules that relate the received data to a likelihood of self-extubation.

In some embodiments, the computer-implemented method may comprise delivering the generated alert signal to a medical professional.

Determining a likelihood of self-extubation by the intubated patient may, in some embodiments, comprise determining that the intubated patient is experiencing a degree of discomfort.

In some embodiments, the data relating to the intubated patient may comprise image data of the patient, data indicative of a motion of the patient and/or of a piece of equipment used in the intubation, data indicative of a sound made by the patient, and/or physiological data of the patient.

According to a second aspect, the present invention provides a computer program product comprising a non-transitory computer-readable medium, the computer-readable medium having computer-readable code embodied therein, the computer-readable code being configured such that, on execution by a suitable computer or processor, the computer or processor is caused to perform steps of the methods disclosed herein.

According to a third aspect, the present invention provides a system for monitoring an intubated patient comprising one or more sensors associated with the intubated patient and a processor configured to: receive, from the one or more sensors, data relating to the intubated patient; determine a likelihood of self-extubation by the intubated patient based on the received data; and responsive to determining that the likelihood of self-extubation is greater than a defined threshold, generate an alert signal.

In some embodiments, the one or more sensors associated with the intubated patient may comprise one or more cameras configured to capture images of the intubated patient.

The one or more sensors associated with the intubated patient may, in some embodiments, comprise one or more wearable sensors, a microphone and/or one or more sensors configured to measure physiological data of the intubated patient.

In some embodiments, the one or more sensors may comprise one or more of an accelerometer, a magnetometer, an oxygen saturation sensor, a capnography sensor, a heart rate sensor, a blood pressure sensor, an electrocardiogram, ECG, sensor and a thermometer.

The system may, in some embodiments, comprise a storage device in communication with the one or more sensors and the processor, wherein the storage device is configured to store data received from the one or more sensors.

These and other aspects of the invention will be apparent from and elucidated with reference to the embodiments described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a schematic illustration of an example of an intubated patient;

FIG. 2 is a schematic illustration of an example of a hand of an intubated patient;

FIG. 3 is a flowchart of an example of a computer-implemented method for monitoring an intubated patient;

FIG. 4 is an illustration of an example of a simulated skeleton corresponding to various body parts of a patient;

FIG. 5 is a flowchart of an example of a computer-implemented method for monitoring an intubated patient;

FIG. 6 is a schematic illustration of an example of a non-transitory computer-readable medium in communication with a processor;

FIG. 7 is a schematic illustration of an example of a system for monitoring an intubated patient; and

FIG. 8 is a schematic illustration of a further example of a system for monitoring an intubated patient.

### DETAILED DESCRIPTION OF EMBODIMENTS

This disclosure relates to methods and systems for monitoring a patient and, in particular, for monitoring an intubated patient. The methods and systems disclosed herein may also be used for monitoring two or more patients simultaneously. A patient may be intubated in a care setting such as a hospital. A patient may be intubated when they are unable to breath on their own and therefore require assisted ventilation. Mechanical ventilation is one type of assisted ventilation. Assisted ventilation may be administered via an endotracheal tube placed into a person's windpipe via their mouth or nose. Both procedures enable a supply of gas, such as an oxygen-containing gas, to be delivered to the patient's lower airways.

It is known that an intubated patient undergoing assisted ventilation can experience discomfort, and intubation can result in the patient becoming restless and/or agitated. Indications that the patient is experiencing discomfort may include movement of various parts of the patient's body, such as their head, arms, hands, legs, feet, and/or torso. Other indications that the patient is experiencing discomfort may include changes in their facial expression, sounds made by the patient and/or changes in physiological data relating to the patient, such as data measured using medical equipment. It has been established that an intubated patient who is experiencing high levels of discomfort is more likely to attempt to remove the tube themselves than an intubated patient who is comfortable. Thus, signs of discomfort of an intubated patient may be indicative of an increased likelihood of the imminent occurrence of a self-extubation event.

Embodiments disclosed herein provide a mechanism by which data associated with or relating to an intubated patient may be acquired, analyzed and used to determine a likelihood that the intubated patient will attempt to remove the tube from his or her airway, or self-extubate. If it is determined that the patient is likely to attempt to remove the tube, then appropriate action may be taken to prevent the self-extubation attempt and/or to comfort the patient, thereby reducing the likelihood of self-extubation. For example, an alert may be generated and sent to a medical professional warning them that the patient is experiencing high levels of discomfort and indicating that there is a strong likelihood that, unless action is taken, the patient will attempt to remove the tube.

To assess whether a patient is experiencing discomfort, the patient may be monitored using one or more sensors and/or detectors including, for example, one or more of a camera, an accelerometer, a magnetometer, an oxygen saturation sensor, a capnography sensor, a heart rate sensor, a blood pressure sensor, an electrocardiogram (ECG) sensor, a thermometer and a microphone. A patient's facial expression and motion of their head, one or more arms, one or more hands, one or more feet and/or torso may be analyzed using a camera. It is also envisaged that a combination of multiple sensors, such as those mentioned above, may be used to monitor the patient. Data acquired by the sensors may be referred to as sensor data.

Sensor data may be used alone or in combination to determine a level of patient discomfort. Before an intubated patient attempts to remove a tracheal tube from their airway, the patient may exhibit signs or indications of discomfort, for example, a change in heart rate, heart rate variability, a change in temperature, a particular facial expression, and/or motion of a body part. The sensors associated with the patient may be used to acquire data indicative of one or more of these signs of discomfort, and the acquired data may be provided as an input to a model in order to determine a likelihood that the patient will self-extubate. One such model may be a rule-based model. A rule-based model may employ a rule-based classifier to determine a likelihood of a patient self-extubating. Examples of rules include increasing a likelihood of self-extubation if the patient's head moves beyond a defined threshold through a particular motion (e.g. from measurements of the roll, pitch and/or yaw, or displacement of the head of the patient), or if the patient moves a hand to a position between their torso and head. In other examples, a rule may be based on a pattern and/or frequency of a motion of a body part of the patient; for example, if a patient moves their head from side to side repeatedly within a defined time period. In other examples, a trained predictive model may be used to determine a likelihood of self-extubation. Examples of trained predictive models include trained neural networks, such as convolutional neural networks, decision trees, support vector machines (SVMs), and the like.

As well as being used for predicting the likelihood of a patient self-extubating, the sensor data may be used more generally for creating a patient comfort indicator.

If the predicted likelihood of a patient self-extubating exceeds a predefined threshold value, then an alert signal may be generated. An alert signal may comprise, for example, a data signal or an instruction, which may be used to sound an alarm, or data forming a message to be delivered to a medical professional. In some examples, the alert may include the likelihood of a patient self-extubating and a time that patient self-extubation is predicted to occur.

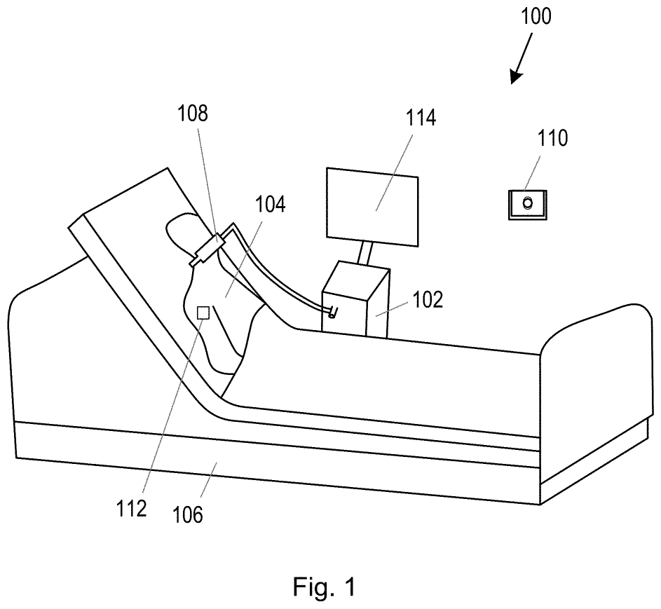

Referring to the drawings, FIG. 1 is an illustration 100 of an intubated patient 104 in a care setting, such as a hospital, lying on a bed 106. The patient 104 is undergoing assisted ventilation, whereby a supply of gas is being administered to the patient via an endotracheal tube 108. The supply of gas is provided by a gas cylinder (not shown). A device for capturing images, such as an imaging sensor, or imaging device (e.g. a camera) 110 may be used to monitor the patient 104 by capturing a series of images (e.g. a video stream) of the patient. The imaging device 110 may, for example, be mounted high on a wall or on the ceiling, overlooking the length of the bed and the patient 104. From the images, a facial expression of the patient 104 may be determined or identified, and/or motion of a body part of the patient may be detected. In some examples, two or more imaging devices may be used to monitor the patient 104. A wearable sensor 112 may be attached to the patient 104 and used to measure data relating to the patient, such as a motion of a body part of the patient, sounds made by the patient and/or physiological data relating to the patient. In some examples, two or more wearable sensors may be attached to the body of the patient 104. The two or more wearable sensors may be attached to the same body part or to different body parts of the patient 104.

A processor 102 may be configured to receive sensor data from sensors 110, 112. The processor 102 may be used to process the sensor data in order to determine the predicted likelihood of the patient 104 self-extubating. The processor 102 may be a standalone component, i.e. a separate component to the sensors 110, 112, or may form an integral part of one of the sensors. If the processor 102 is a separate component to the sensors 110, 112, the processor may be located in the same room as the patient 104, the same building as the patient, such as a server room or other onsite location, or at a remote location thereby enabling cloud-based computing functionality. The sensor data may be transmitted to the processor 102 via a wired or wireless connection. Alternatively, one or more of the sensors 110, 112 may have a processor 102 integrated therein, which may support onboard edge computing thereby enabling the sensor data to be processed at the location of the sensors. The predicted likelihood of the patient 104 self-extubating and/or a patient comfort indicator may be displayed on a patient monitor 114 and/or stored in the patient's medical record.

The imaging device 110 may comprise, for example, an image capture device, a camera, a video camera, or a pan-tilt-zoom (PTZ) camera which can be pointed (e.g. at multiple patients) under software control, which in turn may comprise one or more image sensors such as a complementary metal oxide semiconductor (CMOS) or a charge coupled device (CCD). The imaging device 110 may be wall-mounted, positioned on a piece of equipment such as the bed 106 on which the patient 104 lies, the patient monitor 114, or the endotracheal tube 108 connected to the patient. The imaging device 110 may be capable of monitoring a single patient 104 or multiple patients simultaneously. Preferably, the field of view of the imaging device 110 will include at least part of the patient 104 and, preferably, the whole of the patient. An array of imaging devices 110 may be employed.

The processor 102 may apply a computer vision algorithm to the collected sensor data from the imaging device 110 to determine, for example, a facial expression of the patient 104. The facial expression of the patient 104, as determined using the computer vision algorithm, may be used as an input to the rule-based model and/or the trained predictive model, as mentioned briefly above, and discussed in greater detail below, to determine the likelihood of the patient self-extubating.

Various types of wearable sensors may be used to acquire data relating to the patient 104 including, for example, an accelerometer, a magnetometer, an oxygen saturation sensor, a capnography sensor, a heart rate sensor, a blood pressure sensor, an electrocardiogram (ECG) sensor and a thermometer.

In some embodiments, an accelerometer may be used to measure acceleration forces. An accelerometer may be located on, for example, a body part of the patient 104, such as their head, arm, hand, foot or torso, or may be located on the endotracheal tube 108 associated with the patient. An accelerometer measurement may be used to determine a motion of the body part of the patient 104 to which it is attached. A change in the motion of a body part of the patient 104 may be indicative of a change in a level of discomfort of the patient. In some cases, for example, a pattern, frequency and/or magnitude of the motion may be associated with the level of discomfort of the patient 104. For example, motion detected using an accelerometer located on the endotracheal tube 108 associated with the patient 104 may be indicative that the patient is moving their head or is pulling on the tube. A high level of discomfort may be associated with the patient 104 moving their head from side to side rapidly, whereas a low level of discomfort may be associated with the patient keeping their head relatively motionless.

In some embodiments, one or more of the wearable sensors 112 may comprise a magnetometer, which is a device that measures the direction, strength and change in a magnetic field at a particular location. A magnetometer may be used in combination with a gyroscope and/or an accelerometer in order to determine, for example, a force and/or orientation of the device. A magnetometer may, in some examples, be employed within an inertial measurement unit. A measurement of force and/or a change in orientation, as measured using a magnetometer and/or inertial measurement unit, may be indicative of a motion of the patient 104 and, therefore, the level of discomfort of the patient.

In some embodiments, an oxygen saturation sensor may be used to measure an oxygen saturation level of the patient 104. The oxygen saturation level of the patient 104 may be measured using, for example, a pulse oximeter. Pulse oximetry may be performed invasively or non-invasively. A non-invasive pulse oximeter may be attached to a finger of the patient 104, for example. A particularly high or low oxygen saturation level, or a sudden change in oxygen saturation level, may be indicative that the patent 104 is experiencing higher levels of discomfort and, therefore, may be more likely to self-extubate.

In some embodiments, a capnography sensor may be used to determine a concentration of carbon dioxide in a breath of the patient 104. The capnography sensor may be located on the endotracheal tube 108 associated with the patient 104. Particularly high or low levels of carbon dioxide in the patient's breath, or a sudden change in carbon dioxide levels, may be indicative that the patent 104 is experiencing higher levels of discomfort and, therefore, may be more likely to self-extubate.

In some embodiments, a heart rate sensor may be used to measure a heart rate of the patient 104 and may be located on the body of the patient, such as their arms, hands, feet, or torso. A pulse oximeter may be used to measure the heart rate of the patient 104. The heart rate of the patient 104 may be associated with the level of discomfort of the patient. For example, a relative increase in the heart rate of the patient 104 may be indicative that the patient is experiencing a relatively increased level of discomfort.

A blood pressure sensor may, in some embodiments, be used to measure a blood pressure of the patient 104 and may be located on the body of the patient, such as an arm. For example, an increase in the blood pressure of the patient 104 may be indicative of an increase in the level of discomfort of the patient and, therefore, an increased likelihood that the patient will self-extubate.

In some embodiments, an electrocardiogram (ECG) sensor may be used to measure electrical activity of the heart of the patient 104 as a function of time. Electrodes may be attached to the body of the patient 104, such as an arm, hand, leg, foot and/or torso. ECG sensor measurements may be used to measure changes in parameters such as the heart rate, inter-beat interval and/or heart rate variability of the patient 104. These parameters may be associated with an emotional and/or physical state of the patient 104 and, therefore, may be associated with the level of discomfort of the patient.

A thermometer may be used, in some embodiments, to measure a temperature of the patient 104, such as the temperature of the skin, or the inside of an ear or mouth of the patient. A change in temperature of the patient 104 may be indicative of a change in the level of discomfort of the patient. For example, an increase in temperature of the patient 104 may be associated with an increased level of discomfort, whereas a decrease in temperature may be associated with a decreased level of discomfort.

In some embodiments, a microphone may be used to measure sounds made by the patient 104. A microphone may form a part of the imaging device 110 or may be a separate component located, for example, on a piece of equipment located within the room in which the patient 104 is being treated, such as the bed 106, patient monitor 114, or the like. Sounds generated by the patient 104 such as speech, groaning, or other sounds generated by the vocal tract of the patient, may be indicative of a level of patient discomfort. For example, if the patient 104 is groaning, then this sound may be associated with an increase in the level of discomfort of the patient. Similarly, sound associated with patient motion may also be detected and may be indicative of patient discomfort.

The level of discomfort of the patient 104 as determined by any of the sensors 110 or wearable sensors 112 may be used to predict the likelihood that the patient will self-extubate. In some embodiments, the data from multiple sensors may be algorithmically fused in order to predict a likelihood of patient self-extubation.

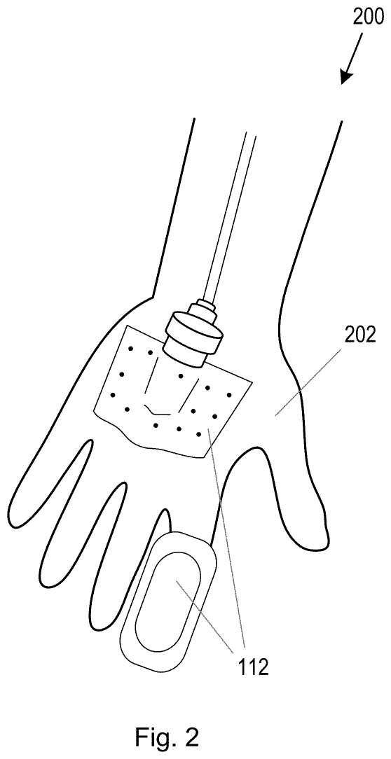

FIG. 2 is an illustration 200 of a hand 202 of the patient 104 (the hand 202 is not visible in FIG. 1) showing example positions of various sensors. One or more wearable sensors 112 may be attached to the hand 202 of the patient 104, and used to measure a motion of the hand 202 and/or physiological data of the patient. In the example shown in FIG. 2, a pulse oximeter is attached to the patient's finger and a motion sensor (e.g. including an accelerometer and/or a magnetometer) is attached to the back of the patient's hand. In some examples, an accelerometer may be integrated with a pulse oximeter.

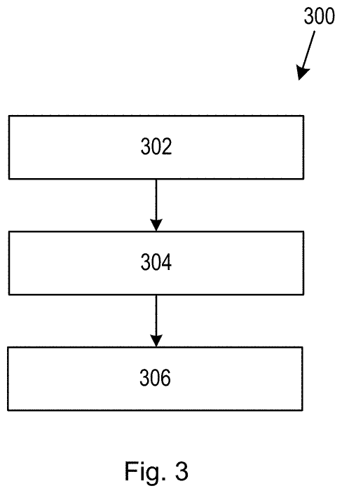

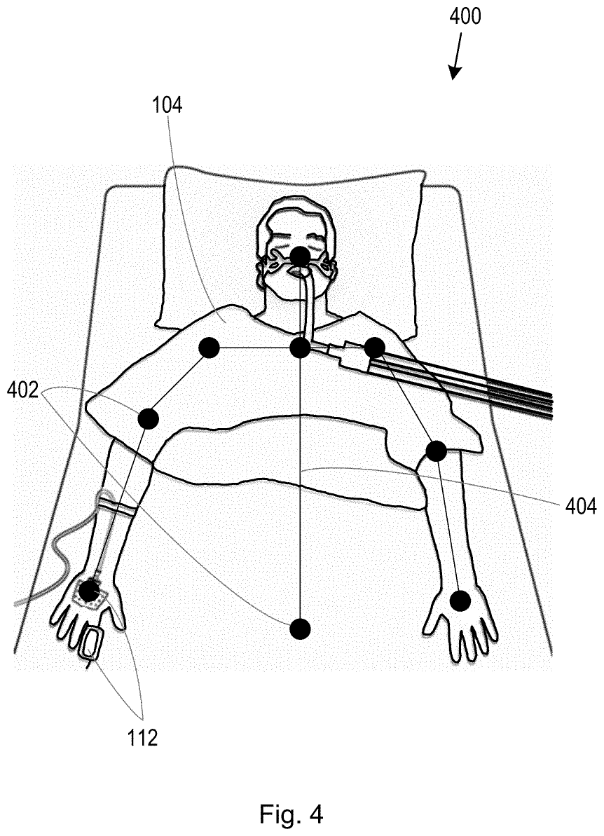

FIG. 3 is a flowchart of an example of a computer-implemented method 300 for monitoring an intubated patient 104. Step 302 of the method 300 comprises receiving, from one or more sensors 110, 112 associated with the intubated patient 104, data relating to the intubated patient. Examples of data relating to the intubated patient 104 may comprise image data of the patient, data indicative of a motion of the patient and/or of a piece of equipment used in the intubation, data indicative of a sound made by the patient, and/or physiological data of the patient. The patient 104 may be monitored using sensors and monitoring techniques such as those discussed above. Step 304 of the method 300 comprises determining a likelihood of self-extubation by the intubated patient based on the received data, as discussed in more detail with relation to FIG. 4 below. Step 306 of the method 300 comprises, responsive to determining that the likelihood of self-extubation is greater than a defined threshold, generating an alert signal.

Step 304 determines the likelihood of self-extubation by the intubated patient 104 based on the received data, i.e. the likelihood that the intubated patient will self-extubate. Step 304 is performed before the patient 104 removes their endotracheal tube (i.e. self-extubates) in order to determine the likelihood that the patient will self-extubate before the event of self-extubation occurs. In some examples, determining a likelihood of self-extubation by the intubated patient 104 may comprise applying a set of rules that relate the received data to a likelihood of self-extubation. Determining a likelihood of self-extubation by the intubated patient 104 may, in some examples, comprise providing the received data as an input to a trained predictive model, wherein the predictive model is trained to determine the likelihood of self-extubation based on the input data. An input into the rule-based classifier and/or the trained predictive model may include a degree or rate of motion of a head, arm, hand, leg or torso of the patient 104, an indication of the patient's facial expression or an image showing the facial expression, or any other measurement from the sensors 110, 112. A processor, such as processor 102, may perform step 304 using a rule-based classifier or a trained predictive model in order to determine the likelihood of the patient 104 self-extubating.

A likelihood of self-extubation may, in some embodiments, be provided in the form of a value ranging from zero to one, such that a value of zero corresponds to a patient who is not predicted to self-extubate and a value of one corresponds to a patient who is predicted to self-extubate. For example, a likelihood of self-extubation of 0.5 may correspond to a predicted probability of 50% that a patient will self-extubate. In other examples, the self-extubation likelihood may be provided as a percentage, or in some other form, such as textually (e.g. descriptively) or graphically as an image or visual indication.

In some embodiments, determining the likelihood of self-extubation by the intubated patient 104 based on the received data (step 304), may comprise determining the likelihood of patient self-extubation within a predefined time. For example, the likelihood of patient self-extubation may be a probability, ranging between 0% and 100%, that the patient 104 will self-extubate as a function of time, such as in the next 5 minutes, 30 minutes, 60 minutes, or the like. The likelihood of patient self-extubation may be calculated at any time interval. For example, the likelihood of patient self-extubation may be determined for each minute within the subsequent 30 minute period. In other examples, the likelihood of patient self-extubation may be determined at each five minute interval within the subsequent 30 minute period. In some examples, a time or time period in which patient self-extubation is predicted to occur is determined.

In some embodiments, measuring a motion of the intubated patient may comprise measuring a motion of one or more of a hand, an arm, a foot, a leg, the head and the torso of the intubated patient. Examples of how the motion of some body parts may be measured are discussed with reference to FIG. 4. FIG. 4 illustrates an example of a simulated skeleton 404 corresponding to various body parts 402 of the patient 104. As shown in FIG. 4, a motion of a body part of the patient 104 may be monitored using the imaging device 110 (not shown in FIG. 4) and/or one or more wearable sensors 112. Sensor data from the imaging device 110 and/or a wearable sensor 112 may be received by a processor, such as processor 102 (not shown). The processor may be used to run a program or application known as a “skeleton tracker”, which may be used to determine a movement of a body part or a joint of the patient 104, such as a head, arm, hand, leg, torso, hand, wrist, elbow, shoulder and/or neck of the patient 104. The movement of the body part of the patient 104 may be tracked, for example, by inputting the sensor data obtained from the imaging device 110 and/or the wearable sensor 112 into the skeleton tracker. The skeleton tracker may identify one or more body parts 402 of the patient 104 within an image of a patient and/or a wearable sensor may be associated with a body part of the patient. The skeleton tracker may be used to determine how the position of a body part changes over time. For example, the skeleton tracker may determine a movement of a body part of the patient 104 by quantifying a change in position of the body part between successive sensor measurements of the patient. FIG. 4 illustrates an example of a simulated skeleton 404 corresponding to various body parts 402 of the patient 104.

In some embodiments, a change in a position of a body part of the patient 104 and/or a speed of movement of the body part of the patient may be associated with an increased level of patient discomfort and, therefore, likelihood of self-extubation. Thus, in some embodiments, determining a likelihood of self-extubation by the intubated patient 104 may comprise determining that the intubated patient is experiencing a degree of discomfort. For example, a movement of the torso of the patient 104 may be indicative of the patient moving into an upright position, which may be associated with an increase likelihood of the patient self-extubating. As another example, if the patient 104 moves a hand towards their head, the contribution to the likelihood of self-extubation may increase from, say, 0 to 0.3. As another example, if the patient 104 is observed to move a hand towards their head, and the time taken for that movement is under a defined duration (e.g. one second), then the contribution to the likelihood of the patient self-extubating may increase from 0.3 to 0.4. As a further example, if the patient 104 moves a hand towards their head and moves their head from side to side simultaneously, the contribution to the likelihood of self-extubation may increase from 0.3 to 0.5. In other examples, a likelihood of the patient 104 self-extubating may be based on a movement of a head, arm, hand, leg, foot and/or torso, or a combination of movements, of the patient. In addition to the change in likelihood from the detected motion of the part(s) of the patient 104, data from other sensors may also be analyzed concurrently, and such data may also affect the likelihood of self-extubation.

In some embodiments, measurement of roll, pitch and/or yaw motion of the patient's head, or displacement of the head of the patient 104, may be used to determine a likelihood of the patient self-extubating. For example, if the pitch of the head of the patient 104 is observed to change by more than 15 degrees, the likelihood of self-extubation may increase by an amount, say, 0.1. In some examples, the likelihood of the patient 104 self-extubating may be determined by measuring a frequency of the change in the head position/orientation of the patient. For example, a patient 104 rolling their head rapidly from side to side may be in a large amount of discomfort and, therefore may be more likely to attempt to self-extubate.

In some embodiments, a change in a roll, pitch and/or yaw of a head of the patient 104 is required to change by a minimum threshold amount within a minimum threshold time, to effect a change in the likelihood of patient self-extubation. For example, if the pitch of the head of the patient 104 is observed to change by more than 5 degrees within a 1 second time period, the likelihood of patient self-extubation may increase. In contrast, if the roll, pitch and/or yaw of the head of the patient 104 changes relatively slowly, for example, by less than 5 degrees within 1 second, it may be assumed that the movement is not associated with a change in the level of discomfort and, therefore, the likelihood of patient self-extubation may remain unchanged. A rapid change in the roll, pitch and/or yaw may be indicative of a shaking or shuddering motion of the patient 104. A rapid change in the roll, pitch and/or yaw may be associated with an increased level of patient discomfort and, therefore, increased likelihood of patient self-extubation. For example, if the roll of the head of the patient 104 is observed to increase and decrease repeatedly by more than a defined threshold value, such as 10 degrees over a defined time period, such as 10 seconds, the patient may be moving their head from side to side rapidly. A shuddering or shaking motion of the patient 104 may be assigned a higher likelihood of patient self-extubation than a patient who is relatively motionless.

In some embodiments, a movement of a body part of the patient 104 may have to satisfy a minimum threshold distance before a change in the likelihood of self-extubation changes. For example, a change in the likelihood of the patient 104 self-extubating may occur only if the patient moves a hand to a position between the patient's torso and head; such a motion may be indicative of the patient moving their hand towards their endotracheal tube in order to pull it out. In contrast, no change in the likelihood, or even a reduction in the likelihood, may occur if the patient moves a hand to a position below their torso.

It will be appreciated that the example likelihood values discussed herein are merely examples, and the actual effect of the various sensor measurements may be selected according to the expected effect of a particular measurement.

In some embodiments, a facial expression of the patient 104 may be used to determine a likelihood of patient self-extubation. A facial expression of the patient 104 may be determined by an analysis of a facial landmark of the patient, such as parts or all of one or more eyes, one or more eyebrows, the nose and/or the mouth. A change in the position of one or more of the facial landmarks may be indicative of motion of the patient and, therefore, of patient discomfort. The change in position of a facial landmark may be determined by comparing two images of the patient 104, where the two images are taken at different points in time. The images of the patient 104 may correspond to images taken adjacently in time or, alternatively, a reference image of the patient 104 may be used, such as when the patient was first admitted to hospital. For example, if the patient 104 appears to be smiling, the likelihood of self-extubation may decrease. As another example, if the patient appears to be grimacing, the likelihood of self-extubation may increase. To determine whether the patient 104 appears to be smiling or grimacing, the mouth of the patient may be analyzed and compared to an earlier image of the patient, for example. As another example, one or more eyes of the patient 104 may be analyzed. A lower likelihood of patient self-extubation may be associated with a patient who has their eyes closed, whereas a higher likelihood of patient self-extubation may be associated with a patient who has their eyes open. A relatively higher likelihood of patient self-extubation may be associated with a patient who has both their eyes and mouth open, for example.

The facial expressions of a patient 104 may be determined, in some embodiments, by comparing an image of the patient's face with a set (e.g. in a database) of known facial expressions. For example, such a database may be stored in a storage medium accessible by the processor performing the analysis. In other examples, the facial expression of the patient 104 may be determined using a trained predictive model or classifier. In such an example, an image of the patient's face may be provided as an input to the trained model, and the trained model may provide as an output, the facial expression (e.g. smiling, grimacing, crying) that it considers is most likely based on the image.

In some embodiments, a pain value, fear value and/or comfort value may be used within a rule-based classifier and/or machine learning model. For example, a classifier may be created by applying a machine learning model to a dataset comprising sensor data 110, 112 corresponding to one or more patients along with self-reported degrees of patient discomfort. In some examples, a facial expression classifier may be input into the classifier, wherein the patient's facial expression may be indicative of a level of pain, fear and/or comfort of the patient 104. These values quantify the patient's pain, fear and comfort levels, respectively, which may be expressed as a number between zero and one, a percentage, or some other form, such as textually (e.g. descriptively) or graphically as an image or visual indication. The values of the patient's pain, fear and/or comfort level may translate into a discomfort level of the patient 104 and, therefore, may be used to determine a likelihood that the patient will self-extubate. For example, a pain value greater than 0.4 may correspond to a likelihood of self-extubation of 0.2. A pain value greater than 0.4 combined with a fear value greater than 0.4 may correspond to a likelihood of self-extubation of 0.3. The pain, fear and/or comfort values may be combined with other measurements of the patient 104, such as roll, pitch and/or yaw motion of the patient's head. For example, a pain value greater than 0.4 combined with a fear value greater than 0.4, as well as roll of the patient's head of greater than 40 degrees, may correspond to a likelihood of self-extubation of 0.6. In some examples, the frequency of the change in head position of the patient 104 may be associated with the discomfort level of the patient.

In some embodiments, the step of determining the likelihood of self-extubation by the intubated patient 104 comprises determining the likelihood of patient self-extubation within a predefined time. The likelihood of patient self-extubation may therefore be calculated as a function of time. For example, the likelihood of patient self-extubation may be determined at any time interval, such as for each minute within the subsequent 5 minute, 30 minute, 60 minute period, or the like. In some examples, a time or time period in which patient self-extubation is predicted to occur is determined. In some embodiments, training a neural network may be performed by collecting sensor data, such as image data 110, and categorizing the sensor data into, for example, six classes: experiencing pain, not experiencing pain, experiencing fear, not experiencing fear, experiencing comfort and/or not experiencing comfort. Alternatively or additionally, the sensor data may be assigned values of pain, fear and/or comfort, such as a percentage. For example, a pain value of 100% may correspond to the patient 104 being in agony (i.e. maximum pain), whereas a value of 0% may correspond to the patient being entirely free of pain (i.e. minimum pain).

In some embodiments, a position of a ventilator apparatus associated with the patient 104 may be associated with a level of patient discomfort and, therefore, a likelihood of patient self-extubation. For example, the position of the endotracheal tube 108 associated with the patient 104 may be indicative of a movement of the head of the patient. If the position of the endotracheal tube changes by more than a predefined threshold distance within a predefined threshold time, the likelihood of patient self-extubation may increase. In other examples, a position of an airway adaptor and/or connector of the endotracheal tube 108 associated with the patient 104 may be analyzed to determine a level of patient discomfort. For example, significant movement (e.g. movement exceeding a defined amount, or movement exceeding the defined amount within a defined period of time) may be indicative of patient discomfort. Markers may be placed onto the endotracheal tube 108 to facilitate tracking of the endotracheal tube by the camera 110.

Sensor data from an imaging device 110 and/or a wearable sensor 112 may be analyzed by a processor, such as the processor 102, to determine a temporal pattern within the sensor data. Temporal patterns may be determined using, for example, frequency domain analysis. A temporal pattern analysis may be performed using all sensor data 110, 112. Alternatively, sensor data 110, 112 may be separated into specific time windows, such as a 10 second window, a 20 second window, or the like, and a temporal pattern analysis performed on sensor data falling within each of these time windows.

In some embodiments, sensor data from the one or more imaging devices 110 and/or one or more wearable sensors 112 may be stored on a storage medium for subsequent review and/or analysis. The stored sensor data may be used as training data to train a machine learning model.

Sensor measurements 110, 112 may be input into a trained predictive model. Similarly to the rule-based model, a trained predictive model may analyze the sensor measurements associated with the patient 104 to determine a level of discomfort of the patient and, therefore, a likelihood of self-extubation of the patient. The trained predictive model may be trained using a series of patient images in order to quantify various patient expressions, such as a patient who is experiencing a range of levels of discomfort.

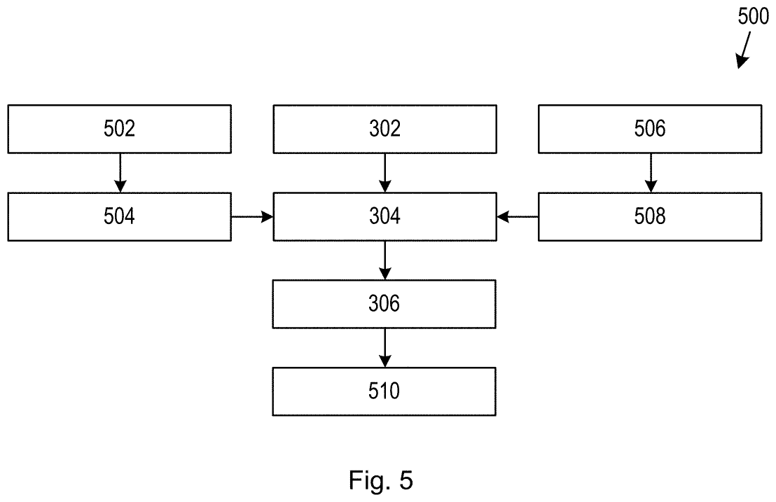

FIG. 5 is a flowchart of an example of a computer-implemented method 500 for monitoring an intubated patient 104. The method 500 may include one or more steps of the method 300 described above. The intubated patient 104 may be monitored using sensors and monitoring techniques such as those discussed above. As noted above, step 302 of the method 300 comprises receiving, from one or more sensors 110, 112 associated with the intubated patient 104, data relating to the intubated patient. The received data may include, for example, images of the intubated patient 104, and may be used in the steps of the method 500.

In some embodiments, the method 500 may include step 502 comprising analyzing a facial expression of the intubated patient 104, and step 504 comprising determining, based on said analysis, that a particular facial expression of the intubated patient is indicative of an increased likelihood of self-extubation. Facial expressions of the patient 104 may be determined using any of the previously described techniques.

In some embodiments, the method 500 may include step 506 comprising measuring a motion of the intubated patient and/or a motion of a piece of equipment used in the intubation, and step 508 comprising, responsive to determining that the measured motion exceeds a defined movement threshold, determining that the measured movement is indicative of an increased likelihood of self-extubation.

Step 304 of the method 300 comprises, as noted above, determining a likelihood of self-extubation by the intubated patient 104 based on the received data, i.e. based on the analysis steps 502 and 504, and/or 506 and 508. As noted above, step 306 comprises, responsive to determining that the likelihood of self-extubation is greater than a defined threshold, generating an alert signal.

In some embodiments, method 500 may further comprise, at step 510, delivering the generated alert signal to a medical professional. For example, an audible alarm may sound to alert nearby medical professionals, or an alert message may be sent to a portable communication device of a medical professional, or to a central monitoring station. Once alerted, a medical professional can take appropriate action to prevent the patient from removing their tube, and/or to calm or comfort the patient, in order to reduce the likelihood of self-extubation. Measuring and analyzing the facial expression and/or the motion of the patient 104 may be performed using any of the previously described techniques.

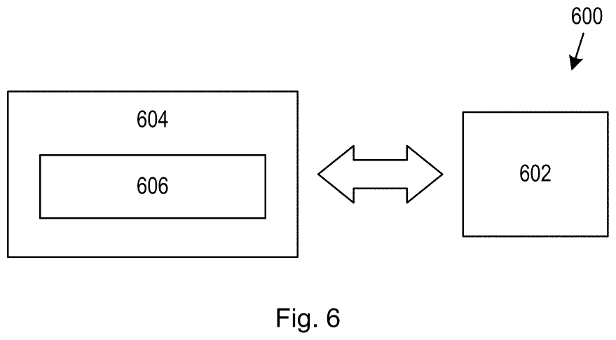

An aspect of the invention relates to a computer program product. FIG. 6 is a schematic illustration of a non-transitory computer-readable medium 604 in communication with a processor 602. The computer-readable medium 604 has computer-readable code 606 embodied therein, the computer-readable code being configured such that, on execution by a suitable computer or processor 602, the computer or processor is caused to perform steps of a method, such as the method 300 and/or the method 500, as described above. The processor 602 may form part of, or be accessible by a computing device, such as a desktop computer, a laptop computer, or the like, or may comprise a server, accessible via a cloud-computing environment.

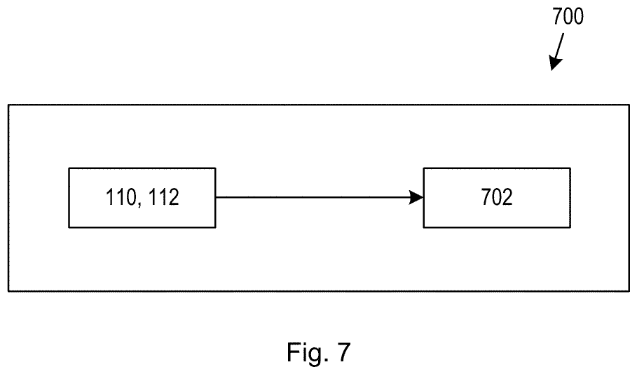

FIG. 7 is a schematic illustration of an example of a system 700 for monitoring an intubated patient 104. The system comprises one or more sensors 110, 112 associated with the intubated patient, and a processor 702. The processor 702 is in communication with the one or more sensors 110, 112. In some embodiments, the processor 702 may be configured to operate or control the sensors 110, 112 and/or one or more other components. The processor 702 is configured to receive, from the one or more sensors 110, 112, data relating to the intubated patient. The processor 702 is also configured to determine a likelihood of self-extubation by the intubated patient 104 based on the received data. The determination may be made using the techniques described herein. Responsive to determining that the likelihood of self-extubation is greater than a defined threshold, the processor 702 may be configured to generate an alert signal. The alert signal may, for example, be delivered to a medical professional so that appropriate action may be taken.

In some embodiments, the one or more sensors 110, 112 associated with the intubated patient 104 may comprise one or more cameras configured to capture images of the intubated patient. The sensors 110, 112 may, in other examples, comprise one or more of the other types of sensor described herein.

In some embodiments, the one or more sensors 110, 112 associated with the intubated patient 104 may comprise one or more wearable sensors, a microphone and/or one or more sensors configured to measure physiological data of the intubated patient. The acquired data may be delivered to the processor 702 for analysis.

In some embodiments, the one or more sensors 110, 112 may comprise one or more of an accelerometer, a magnetometer, an oxygen saturation sensor, a capnography sensor, a heart rate sensor, a blood pressure sensor, an electrocardiogram (ECG) sensor and a thermometer. Data from such sensors may be indicative that an intubated patient is experiencing pain and/or discomfort and, therefore, may be indicative that the patient is more likely to attempt to remove their tracheal tube.

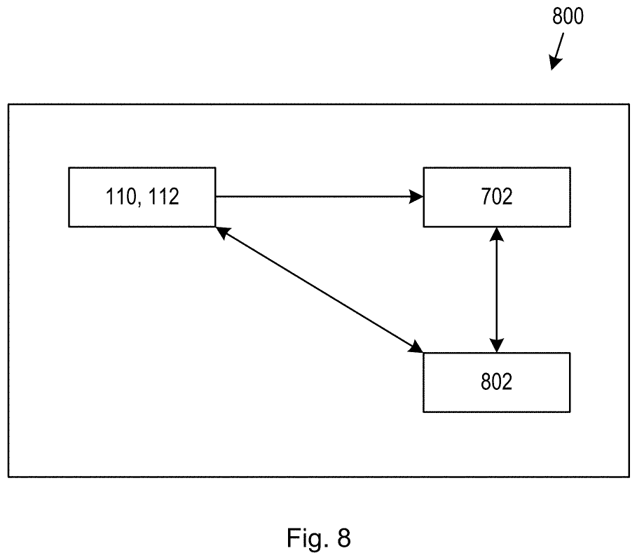

FIG. 8 is a schematic illustration of an example of a system 800 for monitoring an intubated patient 104. In some examples, the system 800 may comprise the one or more sensors 110, 112 associated with the intubated patient 104, the processor 702, and a storage device 802 in communication with the one or more sensors and the processor. The storage device 802 may be configured to store data received from the one or more sensors 110, 112. The stored data may be used by the processor 702 at a later time, for example to analyze various pieces or data and/or to train a predictive model to be used in the method disclosed herein. In addition, calculated self-extubation likelihoods may be stored for review/audit purposes.

The processor 102, 602, 702 can comprise one or more processors, processing units, multi-core processors or modules that are configured or programmed to control the components of the system 700, 800 in the manner described herein. In particular implementations, the processor 102, 602, 702 can comprise a plurality of software and/or hardware modules that are each configured to perform, or are for performing, individual or multiple steps of the method described herein.

The term “module”, as used herein is intended to include a hardware component, such as a processor or a component of a processor configured to perform a particular function, or a software component, such as a set of instruction data that has a particular function when executed by a processor.

It will be appreciated that the embodiments of the invention also apply to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to embodiments of the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computer-implemented method for monitoring an intubated patient, the method comprising:
receiving, from one or more sensors associated with the intubated patient, data relating to the intubated patient;
determining a likelihood of self-extubation by the intubated patient based on the received data; and
responsive to determining that the likelihood of self-extubation is greater than a defined threshold, generating an alert signal.

2. The computer-implemented method of claim 1, wherein determining the likelihood of self-extubation by the intubated patient comprises:
analyzing a facial expression of the intubated patient; and
determining, based on said analysis, that a particular facial expression of the intubated patient is indicative of an increased likelihood of self-extubation.

3. The computer-implemented method of claim 1, wherein determining the likelihood of self-extubation by the intubated patient comprises:
measuring a motion of the intubated patient and/or a motion of a piece of equipment used in the intubation; and
responsive to determining that the measured motion exceeds a defined movement threshold, determining that the measured movement is indicative of an increased likelihood of self-extubation.

4. The computer-implemented method of claim 3, wherein measuring a motion of the intubated patient comprises measuring a motion of one or more of a hand, an arm, a foot, a leg, the head and the torso of the intubated patient.

5. The computer-implemented method of claim 1, wherein determining the likelihood of self-extubation by the intubated patient comprises providing the received data as an input to a trained predictive model;
wherein the predictive model is trained to determine the likelihood of self-extubation based on the input data.

6. The computer-implemented method of claim 1, wherein determining the likelihood of self-extubation by the intubated patient comprises applying a set of rules that relate the received data to a likelihood of self-extubation.

7. The computer-implemented method of claim 1, further comprising:
delivering the generated alert signal to a medical professional.

8. The computer-implemented method of claim 1, wherein determining the likelihood of self-extubation by the intubated patient comprises determining that the intubated patient is experiencing a degree of discomfort.

9. The computer-implemented method of claim 1, wherein the data relating to the intubated patient comprises image data of the patient, data indicative of a motion of the patient and/or of a piece of equipment used in the intubation, data indicative of a sound made by the patient, and/or physiological data of the patient.

10. A computer program product comprising a non-transitory computer-readable medium, the computer-readable medium having computer-readable code embodied therein, the computer-readable code being configured such that, on execution by a suitable computer or processor, the computer or processor is caused to perform the method of claim 1.

11. A system for monitoring an intubated patient, the system comprising:
one or more sensors associated with the intubated patient; and
a processor configured to:
receive, from the one or more sensors, data relating to the intubated patient;
determine a likelihood of self-extubation by the intubated patient based on the received data; and
responsive to determining that the likelihood of self-extubation is greater than a defined threshold, generate an alert signal.

12. The system of claim 11, wherein the one or more sensors associated with the intubated patient comprise one or more cameras configured to capture images of the intubated patient.

13. The system of claim 11, wherein the one or more sensors associated with the intubated patient comprise one or more wearable sensors, a microphone and/or one or more sensors configured to measure physiological data of the intubated patient.

14. The system of claim 13, wherein the one or more sensors comprise one or more of an accelerometer, a magnetometer, an oxygen saturation sensor, a capnography sensor, a heart rate sensor, a blood pressure sensor, an electrocardiogram, ECG, sensor and a thermometer.

15. The system of claim 11, further comprising:
a storage device in communication with the one or more sensors and the processor;
wherein the storage device is configured to store data received from the one or more sensors.
